(19) United States
(12) Patent Application Publication
(10) Pub. No.: US 2016/0262693 A1
(43) Pub. Date: Sep. 15, 2016

(54) METABOLIC ANALYZER FOR OPTIMIZING HEALTH AND WEIGHT MANAGEMENT

(71) Applicant: CASE WESTERN RESERVE UNIVERSITY, Cleveland, OH (US)

(72) Inventor: Amy R. Sheon, Cleveland, OH (US)

(21) Appl. No.: 15/029,383

(22) PCT Filed: Oct. 14, 2014
(86) PCT No.: PCT/US14/60494

§ 371 Date: Apr. 14, 2016

Related U.S. Application Data
(60) Provisional application No. 61/890,854, filed on Oct. 14, 2013.
(60) Provisional application No. 61/891,265, filed on Oct. 15, 2013.

(51) Int. Cl. A61B 5/00; A61B 5/11

(52) U.S. Cl. CPC A61B 5/4866; A61B 5/1118; A61B 5/7282; A61B 5/7475; A61B 5/742; A61B 5/486; A61B 2503/10; A61B 2560/0223

(57) ABSTRACT

A system including a metabolic rate monitor can monitor one or more metabolic determinants to determine a user's metabolic rate. An interval identifier can detect a plurality of intervals corresponding to a least one type of user activity over a time period, wherein each of the plurality of intervals is employed to record the user's metabolic rate determined by the metabolic rate monitor. A metabolic adaptation calculator can determine an adaptation of the user's metabolic rate based on analyzing the user's metabolic rate over each of the plurality of intervals. A recommendation module can provide an output indicating at least one of the metabolic determinants to adjust in response to determined adaptation.

### CROSS-REFERENCE TO RELATED APPLICATIONS

This application claims the benefit of U.S. Provisional Patent Application 61/890,854 filed on Oct. 14, 2013, and entitled METABOLIC ANALYZER FOR OPTIMIZING WEIGHT CONTROL and also claims the benefit of U.S. Provisional Patent Application 61/891,265 filed on Oct. 15, 2013, and entitled METABOLIC ANALYZER FOR OPTIMIZING WEIGHT CONTROL. The entire contents of each of the above-referenced applications are incorporated by reference herein.

### TECHNICAL FIELD

This disclosure relates to metabolic analysis for optimizing health and weight management.

### BACKGROUND

Over the past twenty years, the prevalence of obesity in the general population has skyrocketed. For example, more than one in three adults are obese—meaning that their body mass index is above thirty. Many of the leading causes of preventable death, including heart disease, stroke, diabetes and some forms of cancer are caused by obesity. Diet and weight loss programs provide a reasonable plan to mitigate the effects of such diseases. Unfortunately, weight loss attempts commonly fail because consumers have difficulty achieving energy imbalance required for sustained weight loss.

### SUMMARY

This disclosure relates to metabolic analysis for optimizing health and weight management.

As one example, a metabolic rate monitor can monitor one or more physiologic characteristics to determine a user's metabolic rate. An interval identifier can detect a plurality of intervals corresponding to a least one type of user activity over a time period, wherein each of the plurality of intervals comprises the user's metabolic rate determined by the metabolic rate monitor. A metabolic adaptation calculator can determine an adaptation of the user's metabolic rate based on analyzing the user's metabolic rate over each of the plurality of intervals. A recommendation module can provide an output indicating at least one metabolic determinant to adjust in response to determined adaptation.

As another example a method can include monitoring, by a processor, one or more metabolic determinants to determine a user's metabolic rate. The method can include determining, by the processor, a plurality of time intervals of user activity based on comparing an indication of user activity relative to at least one activity threshold. The method can include analyzing, by the processor, the user's metabolic rate during each of the plurality of time intervals. The method can also include determining, by the processor, an adaptation to the user's metabolic rate based on comparing the user's metabolic rate from the plurality of intervals to a predetermined adaptation threshold.

As yet another example, a system can include a metabolic signature comprised of one or more energy expenditure components (EEC) associated with analyzing a plurality of metabolic determinants that contribute to a user's metabolism or metabolic rate. A metabolic adaptation calculator can monitor the EEC from the metabolic signature to determine an adaptation to the user's metabolism or metabolic rate. A recommendations module can generate one or more recommendations in response to the adaptation determined by the metabolic adaptation calculator.

### BRIEF DESCRIPTION OF THE DRAWINGS

FIG. 1 illustrates an example of a system that employs a metabolic analyzer to assess an individual's metabolic adaptations to changes to nutrition and physical activity and makes recommendations to overcome metabolic adaptations and improve the rate of weight loss for an individual.

FIG. 2 illustrates an example diagram depicting metabolic determinants and their contribution to the generation of energy expenditure components of metabolism.

FIG. 3 illustrates an example of a metabolic signature that employs classifiers to analyze metabolic determinants and to generate energy expenditure components of metabolism.

FIG. 4 illustrates an example device to measure metabolic activity and collect user input data used to classify metabolic components.

FIG. 5 illustrates an example display output for metabolic activity and adaptations.

FIGS. 6-8 illustrate example display outputs for a metabolic analyzer system.

FIG. 9 illustrates an example of a processor that executes computer readable instructions from a memory to provide a metabolic analyzer to determine metabolic adaptations to improve the rate of weight loss for an individual.

FIG. 10 illustrates a method to determine metabolic adaptations to improve the rate of weight loss for an individual.

FIG. 11 illustrates an example of a system that employs a metabolic analyzer to assess an individual's metabolic adaptations.

FIG. 12 illustrates an example diagram depicting a user's calories burned over time and partitioned for interval analysis.

FIG. 13 illustrates an example of a portion of a graph showing a user's calories burned over time relative to activity thresholds.

FIG. 14 illustrates an example adaptation that occurs to a given physical activity.

### DETAILED DESCRIPTION

This disclosure relates to metabolic analysis for optimizing weight control. As disclosed herein, a metabolic analyzer can be utilized to automatically monitor and guide individuals during the course of a weight control or fitness program over a given period of time. The analyzer can be used for weight loss programs, such as to facilitate weight loss, weight gain or weight maintenance. For instance, the analyzer can be utilized for medical research programs, for employee health programs, personal weight control programs, weight loss programs as well as to enable other forms of weight control and/or athletic training programs. These programs can also include support for weight management during pregnancy/lactation or smoking cessation, for example.

By way of example, a metabolic analyzer determines the components of an individual's total energy expenditure and assesses how various metabolic determinants affect energy expenditure, including calorie burn. For instance, the metabolic analyzer can determine an individual's unique metabolic signature based on real time inputs corresponding to various metabolic determinants of an individual's metabolism. Outputs from the metabolic signature can be generated as energy expenditure components that define both the volitional and non-volitional components of the individual's overall total energy expenditure. A metabolic adaptation calculator in the metabolic analyzer can compare output from the metabolic signature to predetermined metabolism thresholds to detect if an adaptation in one or more of the energy expenditure components has occurred over time. If the metabolic analyzer has determined that metabolic adaptation has occurred, then the system can provide individualized recommendations about adjustments that should be made to one or more of the metabolic determinants to counteract the adaptation and optimize the individual's overall metabolic rate toward achieving a desired weight control goal over a period of time. While in many examples herein, the weight control goal is described as weight loss, the systems and methods disclosed herein can be employed for other forms of weight control, such as gain or maintenance or for other health purposes such as supporting smoking cessation, tracking disease progression, predicting disease onset or otherwise monitoring metabolic adaptations for one or more persons, for example.

As a further example, the energy expenditure components can include Resting Energy Expenditure (REE), the thermic effect of eating (ThEE), volitional physical activity thermogenesis (PAT), and non-exercise activity thermogenesis, known as NEAT. Between about 50 and 70 percent of daily caloric expenditure can be accounted for by REE—the energy needed to support bodily functions. The remainder of caloric expenditure is generally spent through ThEE (about 10-15%), volitional exercise (about 0-20%) and NEAT, which accounts for roughly 10% of daily caloric expenditure. Thus, less than about one half of daily caloric expenditure is within volitional control. Variability in this 900-1800 daily calorie expenditure dwarfs volitional efforts that may lead to a 300-500 calorie reduction in intake and 300 calorie increase through physical activity. This unobserved variability commonly undermines weight loss attempts.

In one example, the metabolic analyzer can determine how much of caloric expenditure is occurring through the various metabolic components in substantially real time, such as based on analysis of such expenditure and/or calories burned during one or more selected time intervals. For instance, the selected intervals can correspond to non-volitional energy expenditures, such as during one or more sedentary periods (e.g., sleep or other restful activities) and/or during recovery periods following high activity levels. The selected intervals corresponding to non-volitional energy expenditures thus can be identified over a period time, such as days or weeks. The metabolic analyzer can provide a recommendation (e.g., feedback) that specifies one or more actions to mitigate or counteract metabolic adaptation based on the analysis over a plurality of the selected intervals.

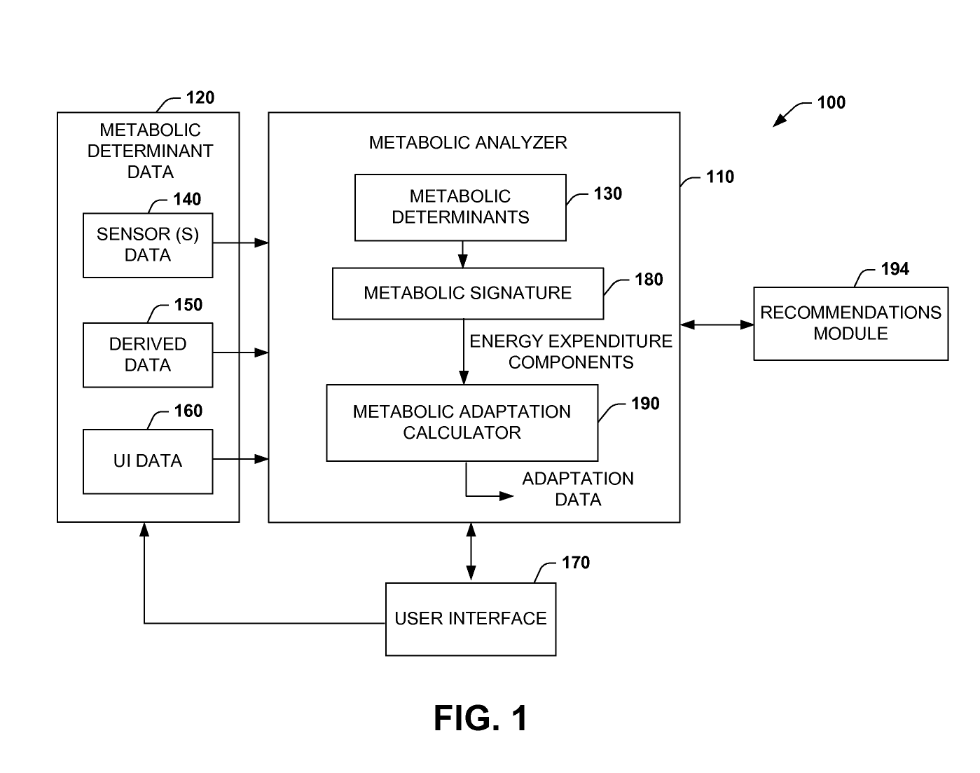

FIG. 1 illustrates an example of a system 100 that employs a metabolic analyzer 110 to determine metabolic adaptations for an individual. The system 100 can be utilized in a variety of weight control programs, such as for weight loss, maintenance or gain or for tracking or predicting other health or performance outcomes. The metabolic analyzer 110 receives metabolic input data 120 that contribute to one or more metabolic determinants 130 that drive various aspects of a user's metabolism. The metabolic determinant data 120 can come from a variety of sources. This can include raw sensor data 140 such as an accelerometer, galvanic skin analyzer, temperature sensor, global positioning sensor, body motion sensor (e.g., accelerometer), indirect calorimetric sensor for analyzing breath, a heart rate monitor, a pedometer, and so forth. The metabolic determinant data 120 can also include derived data 150 which can include data from wearable devices that comprise one or more of the plurality of the aforementioned sensors to generate real time data indicating the user's present metabolism and/or metabolic rate. For example, the derived data 150 can include data representing metabolism or metabolic rate (e.g., calories burned with respect to time, such as kcal/minute) for a given individual. Still yet another source of metabolic determinant data 120 is user interface (UI) data 160 which can be supplied by a user interface 170. For example, the user can indicate when they start and stop eating, changes in medication, when they take medication, when they are hungry, when they are thirsty, when they are resting, and their emotional state. The information in the sensor data, at least some of the derived data and the UI data can indicate or at least affect a change in metabolism and thus can correspond to a metabolic determinant individually or collectively.

The metabolic analyzer 110 utilizes a metabolic signature 180. In one example, the metabolic signature 180 defines a relationship between the metabolic determinants 130 and the energy expenditure components of the user's metabolism over time, such as corresponding to patterns in a given user's energy expenditure. The metabolic analyzer 110 thus can employ the metabolic signature 180 to determine the energy expenditure components in response to the metabolic determinant data 120. Outputs from the metabolic signature 180 are generated as energy expenditure components that define volitional components of the individual's overall metabolism and/or metabolic rate, non-volitional components of the individual's overall metabolism and/or metabolic rate or both the volitional and non-volitional components of the individual's overall metabolism and/or metabolic rate (e.g., REE, ThEE, NEAT and PAT). The metabolic analyzer 110 decomposes the total energy expenditure into the energy expenditure components based on sensor data plus user input data. The metabolic signature 180 defines the relationship between the metabolic determinants and the energy expenditure components.

The metabolic analyzer 110 also includes a metabolic adaptation calculator 190 that is programmed to determine when metabolic adaptations have taken place. The metabolic adaptation calculator 190 can be programmed to compare output from the metabolic signature 180 to one or more predetermined metabolism thresholds to detect if an adaptation in one or more of the energy expenditure components has occurred. For example, the metabolic adaptation calculator 190 can correlate metabolic determinant data 120 for a plurality of analogous types of activities (or inactivity) periods which can be identified in the data over an extended period of time (e.g., days or weeks).

As one example, the metabolic signature can represent data specifying metabolism or metabolic rate (e.g., kcal/min) determined for a plurality of consecutive identified activity periods (e.g., sleep periods, high activity periods, recovery intervals or the like) over an extended time period of days, such as from a selected historical point in time up to the most current data. The metabolic adaptation calculator 190 thus can correlate the metabolic determinant data 120 for each of analogous activity periods to provide adaptation data representing changes in metabolic determinant data 120 over time. If a metabolic adaptation has occurred, the metabolic adaptation calculator 190 can output adaptation data indicating to the metabolic analyzer 110 that an adjustment should be made to one or more of the metabolic determinants 130 to help achieve a goal. For the example of a weight loss goal, the adjustments can be to counteract the adaptation. The adaptation data can initiate individualized recommendations via a recommendations module 194 that is programmed to provide feedback to the individual via the UI 170 on how to improve the metabolic determinants to maximize overall metabolism and thus improve the rate of weight loss over a given period of time. It is noted that various configurations of the system 100 are possible.

By way of further example, all or portions of the system including sensors, metabolic analyzer 110, and user interface 170, and recommendations module 194 could be configured on a wearable device to perform real-time metabolism monitoring and analysis as described herein. Some or all of the processing of the analyzer 110, user interface 170, and/or recommendations module 194 can be provided in accordance with a wireless interface (e.g., Bluetooth) communicating with a cloud storage environment, for example. In other examples, portions of the metabolic analyzer 110, UI 170 and/or recommendations module 194 could be configured to display on a smart phone or other wireless device and communicate (e.g., a wired or wireless link) with devices and/or sensors that process the metabolic determinant data 120.

The metabolic analyzer 110 leverages real-time, full-time monitoring of the rate of caloric expenditure to maximize weight loss or achieve other health goals. This can include the real time visualization of an individual's rate of caloric expenditure. In one example, a wearable device (e.g., an armband) generating the derived data 150 can sense exercise and caloric expenditure, and enable users to enter nutrition intake and details about physical activity via the UI 170. Energy expenditure component data generated from the metabolic analyzer 110 is fed into recommendations module 194 to generate detailed individualized recommendations based on computation of an individual metabolic signature 180. The metabolic signature 180 can also be trained via comparison against corpus of data collected from a plurality (e.g., thousands) of users to further increase the predictive accuracy of the signature.

Recommendations can be offered by the recommendations module 194 in a manner that supports the user's motivation to make and sustain behavior change. Thus, as the user becomes more aware of their on-going metabolism and how it adapts, they can make present modifications which will allow for metabolism gains later in the day such as improvements in their resting energy expenditure (REE). For instance, energy intake can be affected by sleep, thirst, hunger, response to visual cues, and mood. When the overall energy balance is negative meaning that more calories are burned than are ingested, the body undergoes several metabolic adaptations in an effort to conserve energy. Common effects are increased hunger to raise consumption and a reduction in REE as an adaptive effort to maintain a balance between calorie intake and burn. The metabolic analyzer 110 can detect such adaptations via the metabolic signature 180 and guide the user toward improving REE, for example to increase the overall metabolic rate. Recovery is another energy expenditure component that can be determined by the metabolic analyzer 110 and optimized through recommendations if adaptations have been detected.

As noted above, the metabolic determinant data 120 could be derived from any device that includes sensors that monitor and compute caloric expenditure, body movement and sleep time and sleep quality, for example. Sensor input can measure biological parameters such as skin temperature, galvanic skin response, heat flux and acceleration along three orthogonal axes via a three axis accelerometer. Galvanic skin response measures sweat through its increased electric conductivity, an indication of activity. Skin temperature and heat flux also assess the amount of heat generated by the body. The device can determine the amount of energy expended during sedentary periods; through moderate, vigorous and very vigorous activity; and through sleep; average METS (metabolic equivalent) and a number of steps. The device can connect wirelessly to display the user interface 170 on a smart phone or other portable device, such as showing the total calories burned since midnight, total steps, minutes of activity and calories burned during each minute.

The system can also employ a web-based interface 170 programmed for allowing user logging of nutrition intake and body measurements, and links with numerous nutrition and fitness applications. One example links with a display device that can be clipped to clothing for easy reference where the display shows calories burned, steps taken and physical activity duration, for example. Food and activity tracking can include data input by the user of food intake and physical activity. If nutrition information is provided, this can include calculating an up to the moment assessment of energy balance based on the difference between energy consumed and expended. This can also include a capacity to export user data and provide summary reports that can be provided to health care providers or fitness coaches which can also include enabling users to participate in social networks of other users.

The metabolic analyzer 110 can be programmed to analyze both inter and intra-person variations in metabolism. This includes determining components of the individual's metabolic (REE, NEAT, TEE and PAT) via the metabolic signature 180 in comparison to others and then determining their individual response to nutrition, physical activity, sleep, mood, medication and certain elements of blood chemistry. From this analysis, a given individual's “metabolic signature” can be determined for use in computing an indication of the given individual's energy expenditure components and the response to the metabolic determinant data 120. The given individual's metabolic signature can be viewed in comparison with signatures determined from a known population. The metabolic signature can thus be set based on a corresponding classification of the given individual and/or it can be customized based on analysis of metabolic determinants collected for such person during a training phase. The metabolic analyzer 110 thus can provide guidance to help one or more individuals to understand their current metabolic response to nutrition, activity, inactivity, sleep, environmental conditions, and so forth, and then to conduct personally chosen and directed recommendations seeking ways to change their metabolic responses and adaptations that occur through weight loss attempts.

Based on the analysis and monitoring of the user, the metabolic analyzer 110 can provide adaptation data to the recommendations module 194 to generate recommendations. The recommendations module 194 can be part of a local device and/or be implemented remotely, such as via a remote server. Automated recommendations can be generated that enhance existing reports to users, health care providers and coaches by customizing advice to reflect the user's individual metabolic signature 180. Domains of advice can reflect the individual's metabolic response to a variety of monitored determinants, such as one or more of the following: eating which includes timing of eating in relation to a chronological clock; sleep, exercise, last meal, hunger. This can include recommendations based on diet composition (including alcohol consumption) and combinations along with hydration recommendations. Exercise recommendations can includes intensity, duration, time since last food intake, exercise and sleep, time of day, cardiovascular vs. strength exercise, and so forth. Additionally or alternatively, the recommendations can include interaction between nutrition composition and exercise on metabolic rate. Other recommendations can be based on medication, stress, health conditions, and user input of laboratory tests such as thyroid hormone levels, for example.

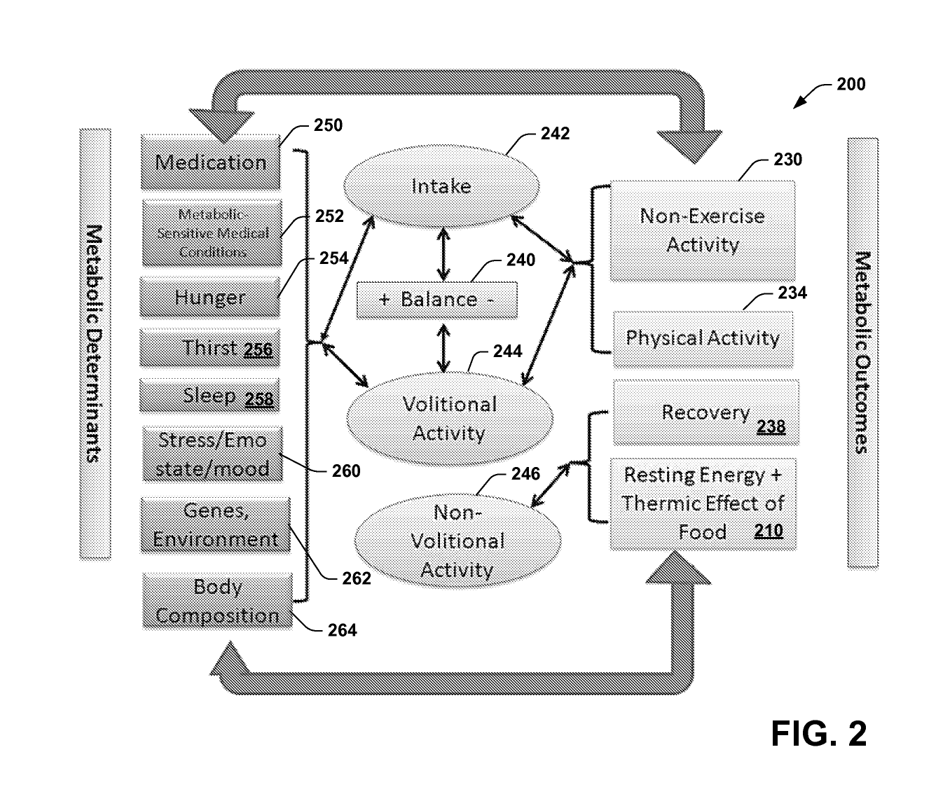

FIG. 2 illustrates an example diagram 200 depicting metabolic determinants and their contribution to the generation of energy expenditure components of metabolism (e.g., depicted as metabolic signature 180). As disclosed herein, the metabolic signature represents information about the individual's rate of energy expenditure (e.g., metabolic rate, such as in kcal/minute) during a set of energy expenditure components (EEC), such as those depicted on the diagram 200. The EEC can include resting energy expenditure (REE) and thermogenic effect of eating (ThEE) 210, non-exercise activity (NEAT) 230 and volitional physical activity thermogenesis (PAT) 234. Together, these components comprise total energy expenditure (TEE). Another component of energy expenditure can include recovery 238, which represents persisting increased metabolic rate after performing exercise, such as resistance training and high intensity interval training, for example.

As shown, energy balance 240 can be computed by the metabolic analyzer 110 and includes a computation of adding intake calories 242 (e.g., amount of calories and composition of fats, carbohydrates and protein) as total caloric intake and subtracting calories burned with respect to volitional 244 and non-volitional activity 246. In another example, the energy balance data can be received as derived data (e.g., metabolic rate) provided to the metabolic signature, such as can be received from a wearable sensor device as described above with respect to FIG. 1. Other metabolic determinants in addition to the caloric intake 242, volitional activity 244, and non-volitional activity 246 include a plurality of factors that can influence the energy balance 240 computation. Such metabolic determinants include the user's medications 250, the user's medical sensitive conditions and associated laboratory values 252, hunger 254, thirst 256, sleep 258, and stress/emotional state 260, for example, which can be indicated in response to the user interfaces described herein. Also, environmental and genetic considerations at 262 and body composition considerations 264 can be included as metabolic determinants. In other examples, one or more sensors can be utilized to monitor one or more of such metabolic determinants 250-264.

The metabolic signature also represents both short term (e.g., minute, hour, day) and longer-term adaptations to EEC occurring over the span of weeks and months. The adaptations can include changes to EEC in response to one or more other determinants, such as hunger, thirst, eating, drinking, not eating, not drinking, exercising, not exercising, sleep, and emotional state such as mood and anxiety, for example. The metabolic signature also includes the individual's experienced hunger and thirst levels in response to nutrition intake, emotional state and sleep, and to the individual's drive to eat and engage in physical activity in response to hunger and TEE.

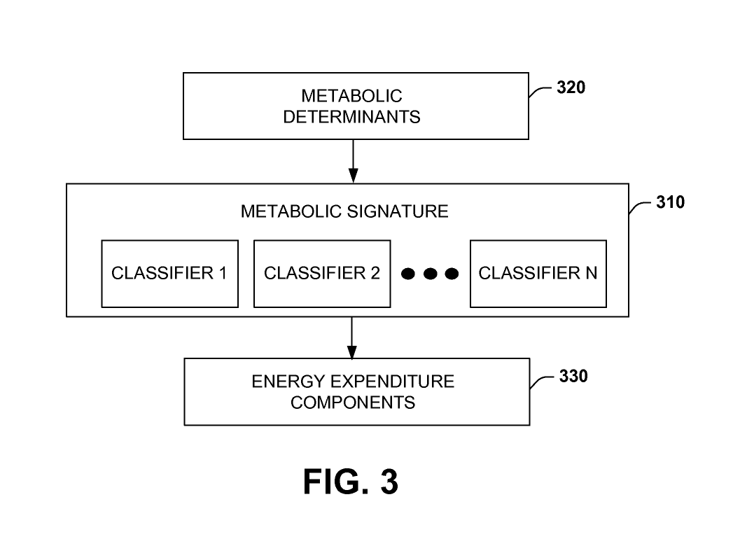

FIG. 3 illustrates an example of a metabolic signature 310 that employs classifiers 1 though N to analyze metabolic determinants 320 and to generate energy expenditure components 350 of metabolism. The classifiers 1-N in the metabolic signature 310 can operate as Bayesian classifiers where machine learning and statistical models can be utilized to determine the energy expenditure components 330. One example of a classifier includes a support vector machine (SVM). Although classifiers are described herein, substantially any type of machine learning can be employed including neural networks, for example. In addition to the classifiers 1-N, other computer executable instructions can be provided to perform the overall functions of the metabolic signature in addition to the metabolic analyzer described above. The following description provides an example of how the metabolic signature can be generated, how machine learning can occur, and other related functions that contribute to the signature determination.

In one aspect for initial training of the signature 310 (e.g., a calibration phase), the user can wear a device monitor for one or more weeks (or other period of time) while making no changes to customary behaviors as far as nutrition and physical activity. In another instance, the user can follow a specified calibration protocol where they undertake specified activities such as sitting for five minutes, eating a high protein meal or walking up a flight of stairs, for example. The user can provide inputs either via buttons on the device or on a smart phone, watch or on a mobile or desktop activity logging application to specify various types of activity or changes in activity. The user input can include goals regarding nutrition and physical activity. For each episode of eating and drinking, then logging of time eating began, time eating ended, food type and quantity consumed can increase the accuracy of the signature. Before and after eating, the user can also input their mood, hunger, and thirst, for example. At specified times of day, the user can also update their mood, hunger, and thirst. For each episode of volitional physical activity (PA), the user can indicate physical activity type, duration, and perceived exertion, for example. The user can specify habitual NEAT activities such as getting dressed, going to work or school, being at work, leaving work, running errands, chores at home, getting ready for bed, and so forth.

Sensors (e.g., either raw sensor data or derived data) can determine hydration level, sleep time and quality, and energy expenditure. The metabolic signature 310 can break down the caloric expenditure of each minute (or other timeframe) into a plurality of metabolic rate components: REE, ThEE, NEAT, PAT, AT. This classification can be determined by an analysis of sensor data coupled with user input. The precision of these classifications can be improved through user input of more details over time. Based on the combination of user input, sensor data and analysis, the metabolic signature 310 can learn to recognize when the individual is resting, eating, drinking or engaged in NEA or PA and the level of exertion. For instance, the metabolic signature 310 can impute the classification of TEE based on knowing of the individual's usual metabolic response to eating, and based on activity patterns. Continuing user input to the signature of when eating begins can increase the accuracy of this calculation. While the above examples demonstrate usefulness of user inputs to specify and help identify types of user activity, in other examples, one or more types of activity can be identified automatically based on sensor data acquired from wearable body sensors without requiring any user interaction. For instance, the identified types of activity can be stored as metadata for associating a respective type of activity to the acquired sensor data that is stored in memory.

Based on the analysis of user input and/or acquired sensor data, the metabolic signature 310 can generate various types of metabolic data. This can include both short term and long term dynamics for the set of metabolic components—the EECs. On one level, the metabolic signature 310 can include a representation of the user's total EE by minute broken down into each EEC. The metabolic signature 310 can include information of the individual's typical EEC in relation to nutrition, PA, NEA, sleep, mood, caloric intake, and so forth. This can be based on analysis of user input and sensor data as described above, and enriched through comparison with other individuals from a larger data library. In other words, this will include their typical TEE in percent (percent of calories consumed by increased EE); and their typical increase in EE by level of exertion, and from NEAT. The signature 310 can also include trend analysis of conditions that make it likely that the individual will deviate from or stick to nutrition and physical activity goals.

The metabolic signature 310 can also include real time predictions of when the individual will engage in nutrition and PA that are consistent with or deviating from goals. Examples include the likelihood that individual will eat, and what they eat, based on hunger, mood, sleep, EEC components and overall energy balance. This can be based on analysis of the user's input and sensor data during the learning period plus real time input and sensor data. Bayesian statistics can be used via the classifiers to take advantage of all prior known information from the individual, which can include learning from a larger group of users as well. The accuracy of these predictions can be enhanced by identifying other individuals in the corpus of data from all users with similar biologic and behavioral parameters, for example, according to user profile data (e.g., age, sex, weight, height as well as known health conditions). A signature thus can be computed for each of a plurality of groups, such as for each classification or category according to user profiles. To increase accuracy for each individual, however, the metabolic signature is computed for each individual and dynamic to respond to ever changing nutritional, physical, psychological and environmental characteristics of each individual user.

For example, the metabolic analyzer described above can be programmed to report patterns or trends in metabolic behaviors. For instance, the metabolic analyzer can identify that, if the person is in an energy deficit of more than 300 calories, they are likely to binge on fast food which can be determined from their signature 310. From this, the metabolic analyzer can initiate recommendations when predetermined metabolic or activity thresholds have been reached that indicate increased likelihood that these deviations from goals will occur. For example, if it has been more than 6 hours since the individual has eaten and their last meal had no protein, then they should eat a high protein meal or snack to reduce hunger and raise REE. Conditions associated with the user following their nutrition and physical activity goals can include being more likely to exercise after having had at least 8 hours of sleep. Additional area of adaptation includes the reduction in NEAT that can occur in response to increased exercise.

In addition to shorter term patterns described herein that can be determined by the metabolic signature 310, longer term patterns can also be analyzed. This includes using longer term monitoring and training to observe trend analyses indicating adaptive thermogenesis. The determination of adaptive thermogenesis can be made as follows: At time 0, during a learning phase, determine a user's EE and EE component as measured by the signature 310 as described above. (The current state EE can be as assessed by having the user wear the device for a week while engaged in usual activity and inputting height, weight, nutrition intake and physical activity, for example, or by completing a designated set of activities as part of a calibration protocol.) The analyzer, in conjunction with the signature 310, can produce a summary of EE such as the following: Resting, eating, NEA and PA, which can be utilized to determine a baseline for the user.

The data acquired during such training phase can be analyzed to produce statistical equations that summarize the fraction of EE attributable to the various components based on height, weight, intake, NEA, PA and sleep duration and quality, for example. From this analysis, the system can produce coefficients of equations that can be used to predict the EE components a given time (e.g., Time 1) based on a new measurement of weight, intake, PA, NEA and sleep. Following a sustained period of weight loss attempts, a second measurement week can be undertaken. The user can again input height and weight, and carefully track nutrition and physical activity for one week. The coefficients from Time 0 will be applied to the sensor and user inputs during week 2 to predict the TEE and components. That prediction can be compared with results provided by the analyzer on Time 1. The difference between the predicted and actual EEC can be considered to be the body's long term adaptation. The following table provides an example that can be employed for training and metabolic analysis:

TABLE 1

| | | | Measurement |
|---|---|---|---|
| Week 1: | End week 1: | Weeks 2-n | period 2: |
| Enter ht, wt day 0 | Enter wt | Restrict | Continue new |
| | | intake, | usual activities; log |
| | | increase | intake, NEA, PA, |
| | | PA and | etc; enter weight at |
| | | NEA | end of week |
| Follow usual | Analyzer reports TEE | Input when | Apply same |
| activities | | possible | calculations as |
| | | | conducted during |
| | | | week 1 to user and |
| | | | sensor input to |
| | | | show total EE and |
| | | | components in |
| | | | numbers and |
| | | | fraction of 100% of |
| | | | total EE. |
| Log nutrition, | Based on user input of | | Apply coefficients |
| intake, hydration, | activity and intake, | | from week 1 to this |
| NEA, PA | metabolic signature | | week's sensor and |
| | classifies Total EE into | | input data to |
| | components and reports | | determine |
| | fraction of Total EE | | predicted EE and |
| | comprised of TEE, NEAT, | | EEC. |
| | PAT and REE. | | |
| Sensors measure | Analysis of sensor and | | Difference between |
| PA, NEA, heat | input data generate | | actual and |
| flow, hydration, | coefficients of equations | | predicted EE and |
| etc | that show the fraction of | | EEC represents |
| | reported intake that is | | adaptive |
| | expended through TEE | | thermogenesis.* |
| | and the absolute number of | | |
| | calories consumed by TEE; | | |
| | similarly, analysis provides | | |
| | the fraction of total EE | | |
| | accounted for by NEAT | | |
| | and PAT and the total | | |
| | number of calories burned | | |
| | through each activity. The | | |
| | system also produces | | |
| | coefficients that describe | | |
| | the relationship between | | |
| | intake and TEE and | | |
| | between NEAT and PAT | | |
| | and measured activity and | | |
| | exercise. REE is the | | |
| | residual. | | |

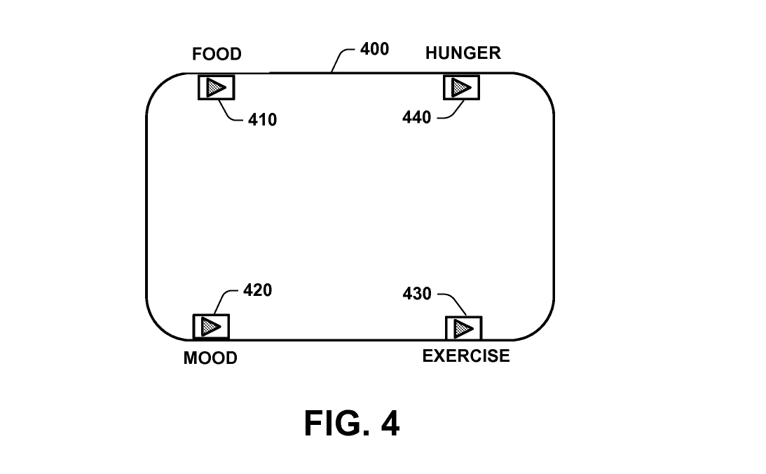

FIG. 4 illustrates an example device 400 to monitor metabolic determinants and display metabolic activity and adaptations. The device 400 can include various buttons to indicate when to start/stop eating at 410, when to start/stop exercise at 420 along with intensity (e.g., multiple pushes for higher intensity). Yet another input 430 (or inputs) can include when the user drinks, is hungry, thirsty, and the current state of their mood, for example. This can also include an input 440 to indicate start/stop/type of exercise, for example. From a web-based interface that communicates with the device 400 (e.g., smartphone or computer), the metabolic analyzer can accept user input of medications and laboratory results related to metabolism. Examples include thyroid levels or other lab tests that might affect a given persons metabolism. Analytic feedback can include daily and real time calorie burn broken down into its constituent components of REE, NEAT, TEE and Volitional exercise, for example, based on user input to the device 400 representing one or more metabolic determinants. The device 400 and/or wireless interface to the device can provide this information in real time, as a graph showing results by the minute, and in summary form (e.g. daily, weekly).

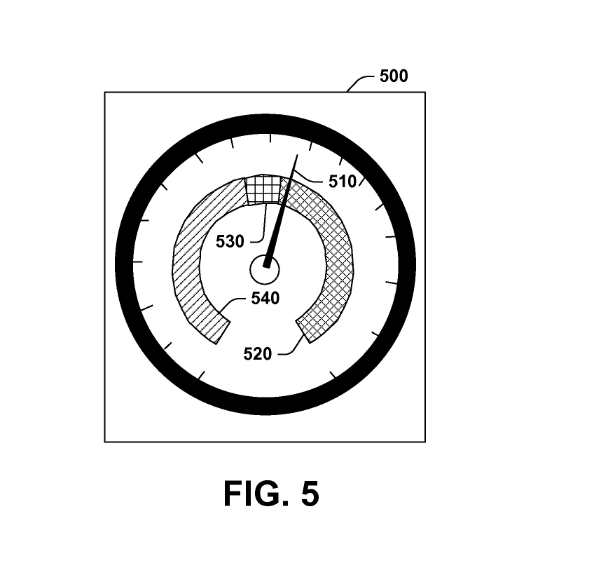

FIG. 5 illustrates an example display output 500 for metabolic activity and adaptations. In this example, the display output can include a metabolic burn rate where the number of calories being burned per minute (e.g., kcal/min), can be shown as a dial 510 that spins faster with faster movement and burn. Different colors 520, 530, and 540 could be used to indicate which illustrated part of the burn is volitional, NEAT, TEE or REE, for example. Audible and/or visual alerts can be generated to the user, such as when hydration or REE drops below predetermined threshold, which can be programmable (e.g., in response to a user input by the user or a health care provider). When hunger data are input as metabolic determinants, the metabolic analyzer can alert users when REE would be optimized by eating or other activity, for example.

User measurements and input as can be stored and encoded/encrypted as Protected Health Information (e.g., HIPAA compliant) to facilitate privacy of the individual supplying metabolic data. Users can be offered a private social network in which they control the privacy of their information with no possibility of data being sold or otherwise made available for marketing purposes, for example.

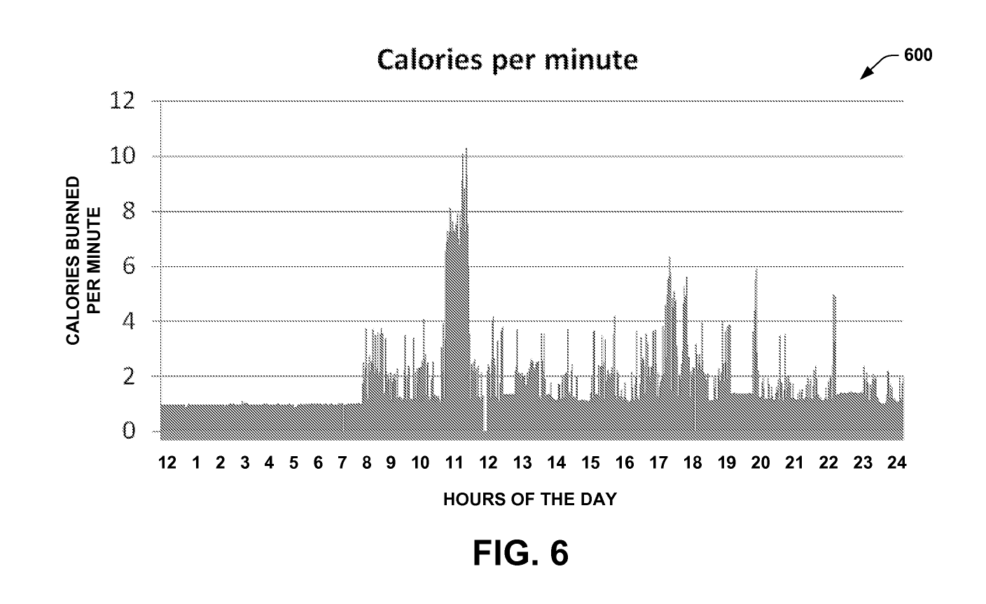

FIGS. 6-8 illustrate example display outputs for a metabolic analyzer system. FIG. 6 shows a graph 600 that depicts calories burned per minute. One or more wearable body sensors (e.g., body motion sensor, pedometer, heart rate sensors, a heat flux sensor, a skin conductance sensor, and a skin temperature sensor and the like) can be employed to automatically specify one or more types of activities or user state information. Additionally or alternatively, the types of activities or user state information can be specified in response to a user input, for example. The types of activities or user state information thus are stored as metadata associated with the caloric burn data from wearable sensor (e.g., generated by caloric expenditure sensor). The graph 600 can show metabolic responses to such inputs.

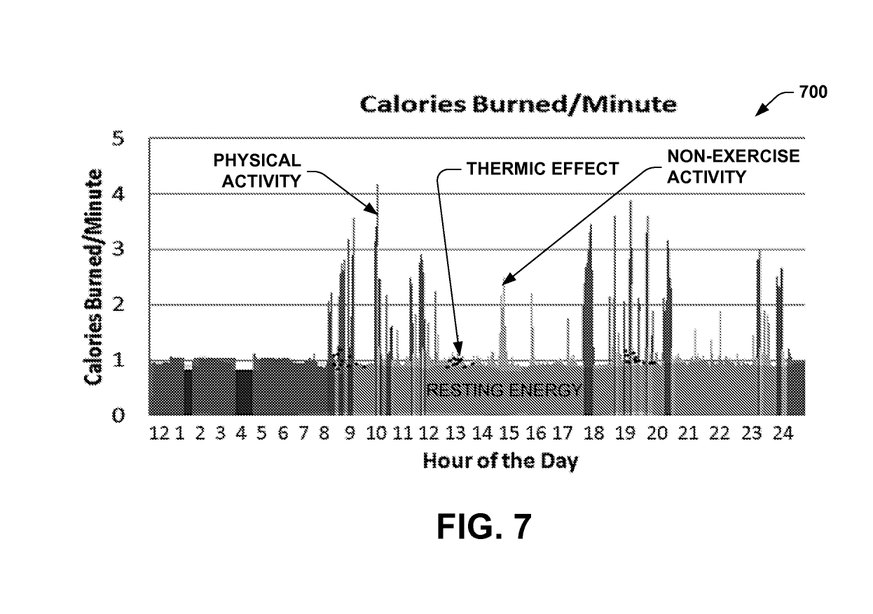

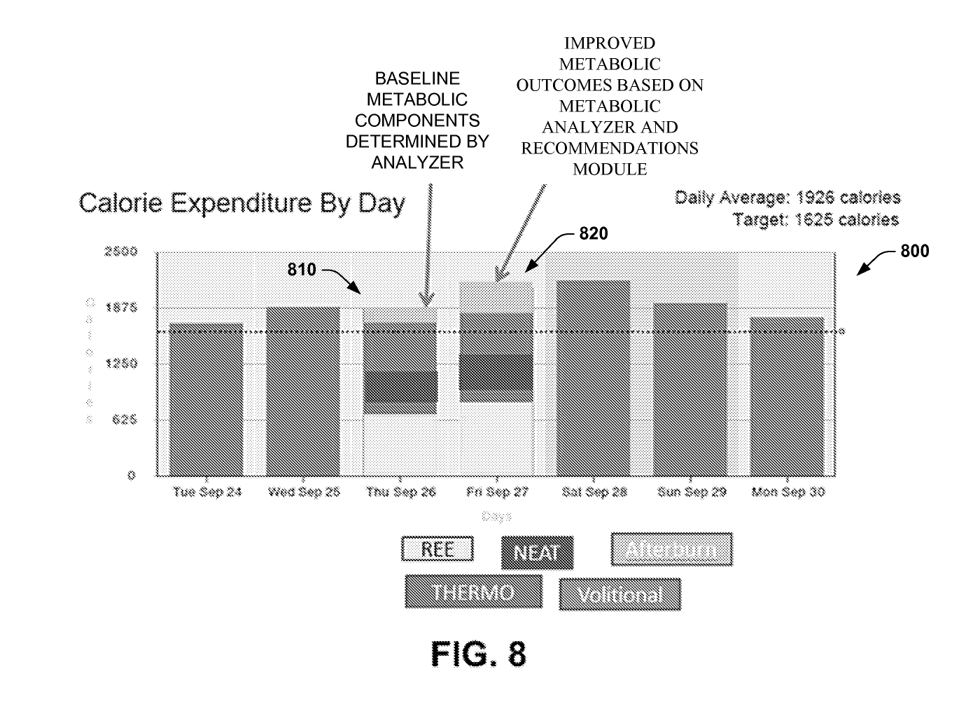

FIG. 7 shows a graph 700 demonstrating different classifications of calorie expenditure by type including resting energy, thermic effect of eating, physical activity and non-exercise activity. Such graph 700 can be employed to motivate the user to help increase REE and recovery, for example. FIG. 8 shows a diagram 800 showing the total daily calorie expenditure broken down and summarized by energy expenditure component such as REE, volitional exercise and thermic effect of food illustrated at 810. After following the metabolic analyzer protocol as described herein, the calorie expenditure can be improved over the baseline performance, such as shown at 820. Notably, REE, volitional exercise, and recovery will generate increased energy expenditure as shown by larger size of various energy expenditure components shown in the graph.

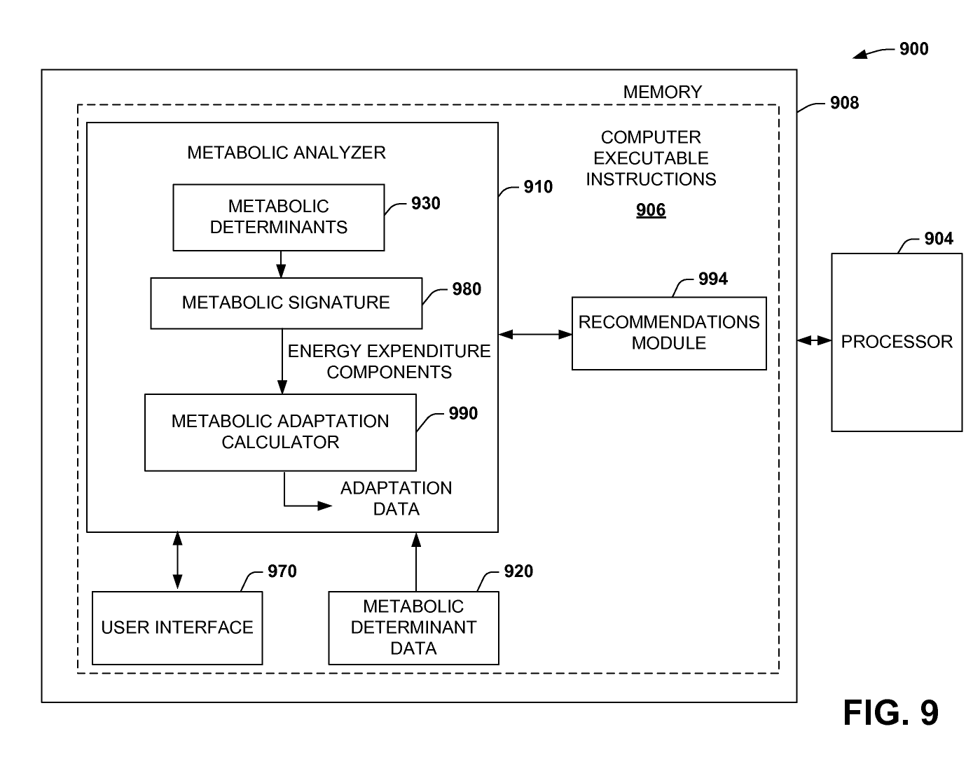

FIG. 9 illustrates an example of a computer system 900 that can implement a metabolic analyzer (e.g., corresponding to analyzer 110) 910 and a recommendations module (e.g., corresponding to module 194) 904, such as disclosed herein. The system 900 includes a processor 904 that executes computer executable instructions 906 from a memory 908 to provide the metabolic analyzer 910 to determine metabolic adaptations to improve the rate of weight loss for an individual. The metabolic analyzer 910 receives metabolic input data (e.g, corresponding to data 120) 920 that contribute to one or more metabolic determinants 930 which drive various aspects of a user's metabolism. The metabolic determinant data 920 can come from a variety of sources as previously described. Still yet another source of metabolic determinant data 120 is user interface (UI) data which can be supplied by a user interface 970. For example, the user can indicate when they start and stop eating, changes in medication, when they are hungry, when they thirsty, when they are resting, and their emotional state which all can indicate a change in metabolism.

The metabolic analyzer 910 utilizes a metabolic signature 980 that monitors the metabolic determinants 930 of the user's metabolism. Outputs from the metabolic signature 980 are generated as energy expenditure components that define both the volitional and non-volitional components of the individual's overall metabolism. A metabolic adaptation calculator 990 in the metabolic analyzer 910 can compare output from the metabolic signature 980 to predetermined metabolism thresholds to detect if an adaptation in one or more of the energy expenditure components has occurred. If a metabolic adaptation has occurred, the metabolic adaptation calculator 990 can output adaptation data indicating to the metabolic analyzer 930 that an adjustment should be made to one or more of the metabolic determinants 930 to counteract the adaptation. The adaptation data can be processed by a recommendations module 994 that provides automated feedback to the individual via the UI 970 on how to improve the metabolic determinants to maximize overall metabolism and/or metabolic rate and thus achieve improved weight control over a given period of time.

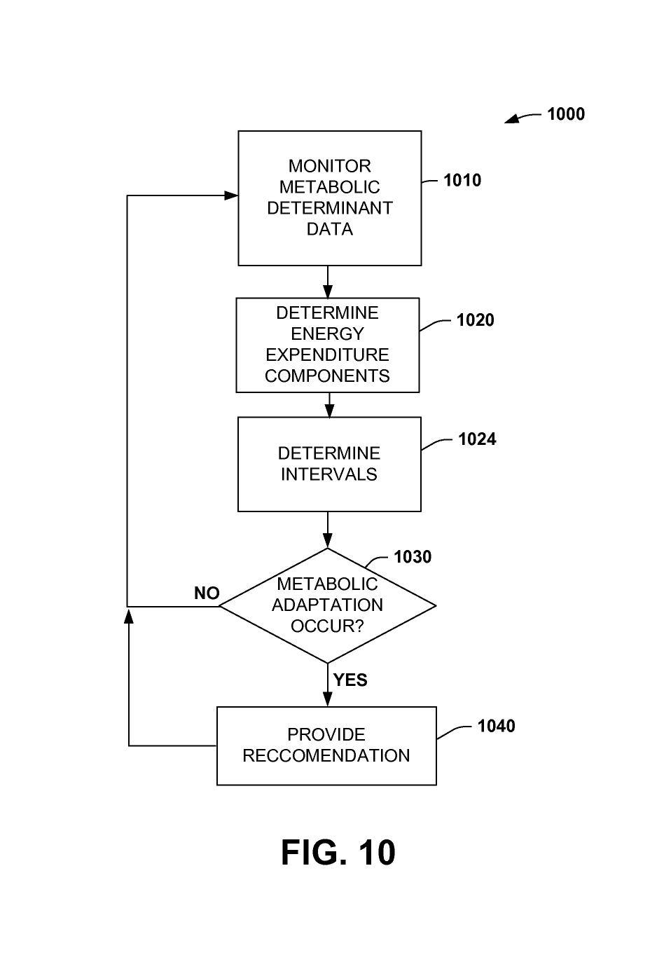

In view of the foregoing structural and functional features described above, a methodology in accordance with various aspects of the present invention will be better appreciated with reference to FIG. 10. While, for purposes of simplicity of explanation, the methodology of FIG. 10 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect of the present invention. The various acts of the method depicted in FIG. 10 can be stored in memory (e.g., memory 908) and executed automatically, such as via a processor (e.g., processor 904), computer, and/or controller configured with executable instructions to carry out the various acts described herein.

FIG. 10 illustrates a method 1000 to determine metabolic adaptations to improve the rate of weight loss for an individual. At 1010, the method 100 monitors metabolic determinant data (e.g., via metabolic analyzer 110 of FIG. 1). At 1020, the method 1000 determines energy expenditure components from the metabolic determinant data (e.g., via metabolic signature 180 of FIG. 1). At 1024, the method includes determining one or more intervals as previously described. At 1030, the method determines whether or not a metabolic adaptation has occurred (e.g., via adaptation calculator 190 of FIG. 1). If no adaptation has occurred at 1030, the method proceeds back to 1010 and continues to monitor metabolic determinant data. If an adaptation has occurred at 1030, the method proceeds to 1040 and supplies a recommendation to counteract the detected adaptation (e.g., via recommendations module 194 of FIG. 1). After supply one or more recommendations at 1040, the method proceeds back to 1010 and monitors metabolic determinant data.

Although not shown, the method 1000 includes monitoring one or more metabolic determinants to determine a user's metabolic rate. This can include determining a plurality of intervals of certain types of activity. For instance, the type of activity or user state can be determined based on user input or determined from data acquired by one or more wearable body sensors. As one example, the method 1000 can determine caloric expenditure intervals that are below a predetermined activity threshold, such as corresponding to sedentary period of sustained inactivity. The method can also include analyzing the user's metabolic rate (calories burned per minute) during each of the plurality of identified intervals. This can include determining an adaptation to the user's metabolic rate recorded over the plurality of intervals by comparing a change of the user's metabolic rate over two or more of the intervals of the plurality of intervals to a predetermined adaptation threshold. The method 1000 can include receiving motion detector data (e.g., from a wearable body motion sensor) to detect when the user activity for a given interval from the plurality of intervals is below the predetermined activity threshold, for example. At least one of the plurality of intervals can be detected from a period of inactivity, such as rest, a period of sleep. Additionally or alternatively, the type of activity or user state can be detected as an interval during recovery period after user activity, for example. The recovery period can be detected after user activity exceeds a predetermined high activity threshold for the user followed by a predetermined low activity threshold for the user, for example. The metabolic rate data (e.g., cal/min) acquired for each of the identified intervals for respective types of activity over a given time period can correspond to a metabolic signature disclosed herein.

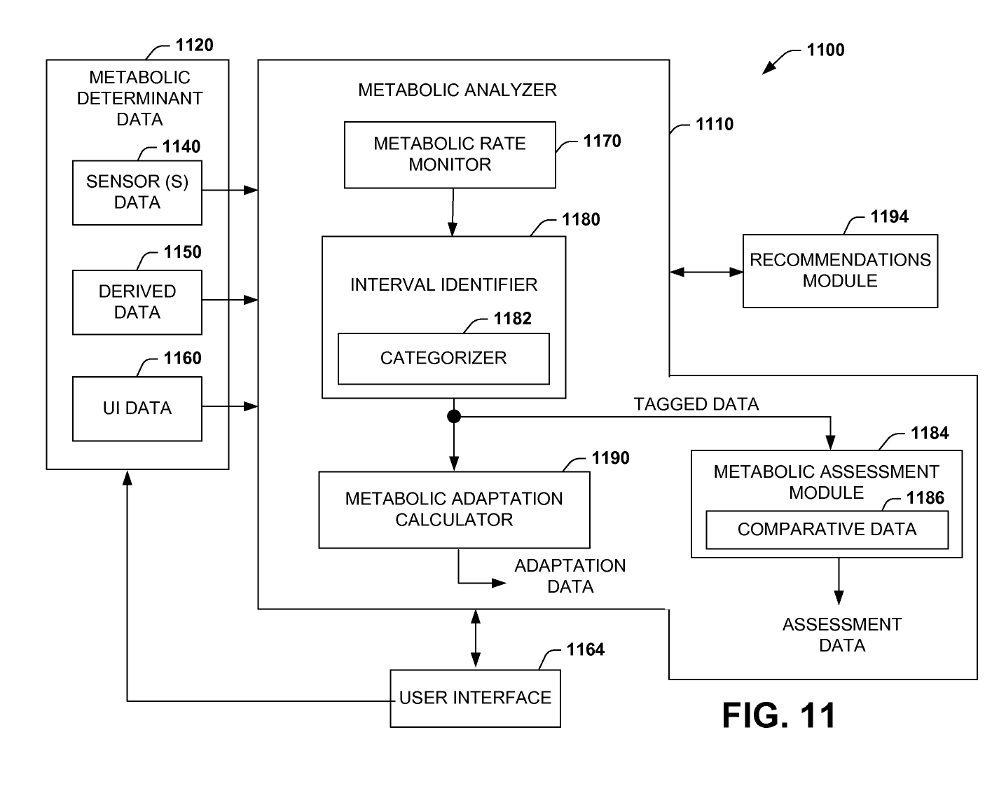

FIG. 11 illustrates an example of a system 1100 that employs a metabolic analyzer to assess an individual's metabolic states and/or adaptations. The system 1100 includes a metabolic analyzer 1110 that analyzes metabolic determinant data 1120 to detect adaptations/state of a user's metabolism or metabolic rate, for example. The metabolic determinant data 1120 can include sensor data 1140 (e.g., motion sensor data, burn rate data), derived data 1150, and user interface data 1160 that can be updated via a user interface 1164. The metabolic analyzer 1110 can include a metabolic rate monitor 1170 to monitor one or more metabolic determinants (described above with respect to FIG. 1) to determine a user's metabolic rate. This can include receiving the metabolic rate (e.g., calories burned per minute) from a wearable device providing sensor data 1140, for example.

An interval identifier 1180 identifies a plurality of intervals of user activity over one or more periods of time (e.g., low activity, high activity, mid-level activity). Each of the intervals can include periods of time associated with a given type of activity or state of the user where burn activity is monitored and/or logged, for example. The interval identifier 1180 includes a categorizer 1182 programmed to identify one or more types of user activity or user state information and assign the types of activity or user state information to the metabolic rate data via tagged metadata (calories burned per minute over selected interval from the monitor 1170). The categorizer 1182 can label different intervals with tagged metadata and output tagged data. The categorizer 1182 can ascertain the type of activity or user state to generate the metadata by the analyzing one or more metabolic determinants, such as disclosed herein.

For example, utilizing time, location, and activity data a given period can be tagged (e.g., user in same location between 11:00 PM and 6:00 AM identifies a sleep period). In some examples, an interval can be detected by the identifier 1180 and then categorized by the categorizer 1182. For example, a low activity interval may be automatically detected when a motion detector senses that motion has ceased (e.g., below a prescribed motion threshold) over a given time frame indicating sleep. Such detected interval can be tagged as sleep by the categorizer for example. In another example, the categorizer 1182 can select a category, such as sleep, and then the interval data is determined based on which data has been assigned such category. As a further example, a user may indicate via the interface 1164 that a category sleep is about to commence. When the user awakes, the user can similarly indicate that the sleep period has ended. The activity can also be inferred automatically based on defining one or more rules based on a set of different determinant data, such as motion (from motion sensor), location (from GPS) and user input data (from user input).

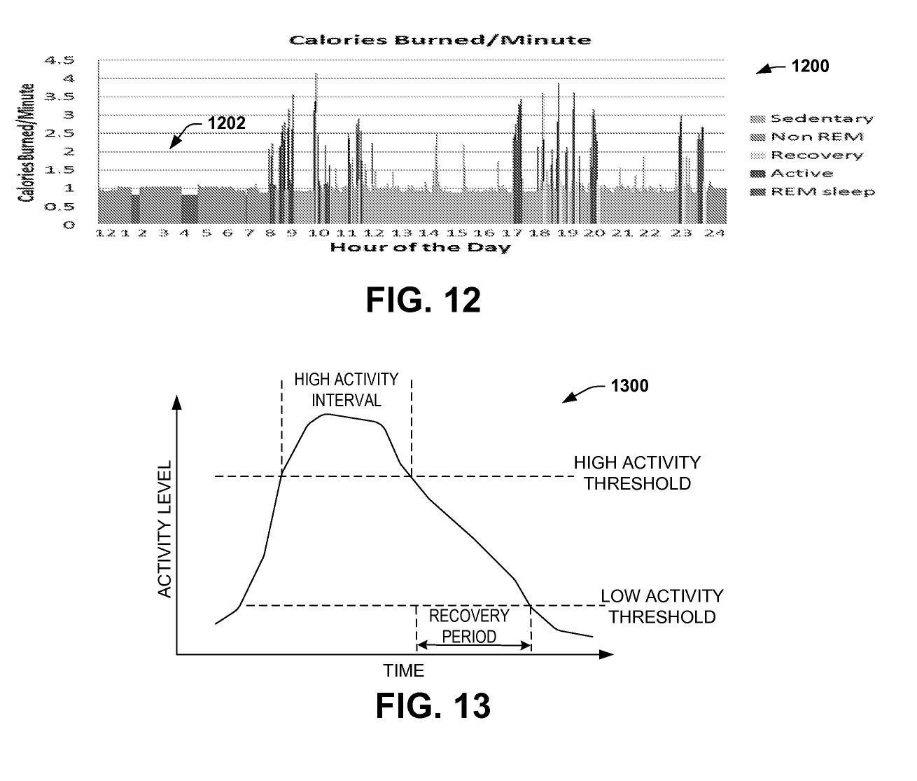

The interval identifier 1180 can specify the interval based on the metabolic determinant data 1120, which can include sensor data 1140, derived data 1150 and/or UI data 1160. Similarly, the categorizer can determine the activity category (e.g., sleep, rest, exercise) sensor data 1140, derived data 1150 and/or UI data 1160. As one example, the interval identifier 1180 identifies one or more intervals based on energy expenditure data (e.g., calories burned per minute) stored in memory from a historic point in time up to the most current acquired data. As one example, the interval identifier 1180 can identify a sedentary type of activity (e.g., sleep or other rest period) by identifying each segment of energy expenditure data (see, e.g., FIG. 12) that is determined to be below a predetermined threshold for a minimum duration of time (e.g., 9 minutes of consecutive activity time or a 3 minute recovery interval). The threshold can be a default value or a user-specific value, such a can be programmed based on a user's own baseline level of energy expenditure.

Additionally or alternatively, data from a wearable body motion sensor that has been acquired concurrently with the energy expenditure data can be utilized by the interval identifier 1180 to identify the activity intervals (e.g., motion detector indicating user is at rest or sleeping). Each of the plurality of intervals can be employed to record the user's metabolic rate determined by the metabolic rate monitor 1170 over time. In response to interval identification, the categorizer 1182 can tag the monitored data per a given category of activity (e.g., sleep, rest, recovery, exercise). For example, the categorizer 1182 programmatically associates metadata to the monitored data specifying one or more categories of activity for each identified interval. The tagged data output from the interval identifier 1180 can be sent to a metabolic assessment module 1184. The metabolic assessment module 1184 can evaluate the tagged data to determine metabolic effects for the determined categories of activity and over the selected intervals. The metabolic assessment module 1184 can perform a variety of analytics, such as compute descriptive statistics, inferential statistics or the like, on the tagged data provided by the interval identifier 1180 (e.g., burn rate/minute data including metadata specifying one or more respective category for each time interval). This can include analysis for specific types/categories of activities and for different periods of time with different metabolic determinants. This can also include analyzing change over one or more and assessment periods and/or categories.

The metabolic assessment module 1184 can also employ comparative data 1186 to assess the user's current metabolism to provide feedback to facilitate the best utilization and adjustment of metabolic determinants. Assessments can include assessments of diet composition, timing of eating in relation to exercise, exercise (timing, duration, intensity, type etc.), timing or duration of sleep, medication, and/or change to health conditions or other lab parameters. Based on such analysis, the metabolic assessment module 1184 enables the user to observe the effects of one or more outcomes including calories burned during sleep, calories burned during sedentary interval, calories burned after eating, and/or rate or duration of recovery, for example. The comparative data 1186 can be employed to compare the user with past data for the user and/or can compare the user with a corpus of other similarly situated users from empirical studies, for example. This can include helping the user select the outcome based on the immediacy or expected duration of the expected effect as well.

A metabolic adaptation calculator (e.g., corresponding to metabolic adaptation calculator 190 from FIG. 1) 1190 monitors the user's metabolic rate recorded over the plurality of intervals and categorized over a selected time period (e.g., including up to the most current data) by the categorizer 1182 to determine an adaptation to the user's metabolic rate. Based on the determination of adaptation by the metabolic adaptation calculator 1190, a recommendations module 1194 can generate one or more recommendations. For instance, in response to detecting a level of adaptation for the user that exceeds a threshold level of metabolic adaptation, the recommendations module 1194 generates recommendations to adjust at least one of the metabolic determinants. The recommendation can be user-specific and differ according to the amount of adaptation that is detected by the metabolic adaptation calculator 1190.

In one example, the interval identifier 1180 evaluates determinant data 1120 (e.g., sensor data 1140, derived data 1150, and/or UI data 1160) capable of indicating when the user's body motion is below the predetermined motion threshold for a plurality of time intervals of a set minimum duration. For instance, an interval can be identified in response to a user activating a button or otherwise indicating they are going to sleep and a motion sensor can confirm that body motion remains below a threshold for at least a prescribed period of time. The same criteria can be applied to determinant data 1120 collected over a plurality of days to identify a plurality of like intervals. As disclosed herein, the identified activity intervals can be selected and tagged by the categorizer 1182 from a period of rest, a period of sleep, a recovery period following a high activity level, a high activity period or other time period which can be uniquely identified and occur repeatedly over a plurality of days (e.g., driving a car to and/or from work, sitting in a meeting or the like).

By analyzing metabolism and/or calorie burn rates during one or more of the different types of identified activity periods (e.g., periods of relative inactivity or activity or recovery intervals), the metabolic adaptation calculator 1190 can determine a level of adaptation associated with one or more of such different types of periods based on changes and trends in the metabolism and/or calorie burn rates. The metabolic adaptation calculator 1190 can also aggregate the determined adaptation from a plurality of different types of tagged activity periods by the categorizer 1182 to provide an aggregate indication of adaptation over a time (e.g., several days or weeks). Periods of inactivity, such as sleep, should prove useful in computing the adaptation since other metabolic determinants affecting metabolism are substantially reduced or absent.

As a further example, a recovery period can be detected after user activity exceeds a predetermined high activity threshold for at least a minimum duration, followed by a rate of energy expenditure that declines at a predetermined rate for a specified period of time, for example (see, e.g., FIGS. 12 and 13). In one example, the metabolic adaptation calculator 1190 determines an adaptation to the user's metabolic rate by comparing a change of the user's metabolic rate over at least two intervals of the plurality of intervals to a predetermined adaptation threshold. For instance, if a burn rate while sleeping drops below a burn rate observed over the prior two weeks while sleeping, it can be determined that a metabolic adaptation has occurred.

In another example, a database (not shown) can be provided where individuals are classified according to groups. Each of the groups can be identified by susceptibility to metabolic suppression, for example, where the adaptation threshold can be varied or selected depending on the relation of a given user to at least one of the groups. A calibration period can be defined for the user to correlate metabolic changes from periods of sleep with activities that occurred during intervals detected when the user is awake, for example. As noted previously, metabolic determinants can be employed to monitor and detect changes with metabolism. The metabolic determinants can include medication input, laboratory values input, hunger inputs, thirst inputs, sleep inputs, food intake, food composition, volitional activity, non-volitional activity, or mood inputs, for example. The recommendations module 1194 can be employed to generate automated recommendations in response to the adaptation data generated by the metabolic analyzer 1110.

Additionally, the metabolic analyzer 1110 can generate a metabolic signature unique for each given user (see, e.g., FIG. 1). The metabolic signature determines a relationship between the metabolic determinants and one or more energy expenditure components that contribute to the given user's unique energy expenditure over time (e.g., an energy expenditure pattern for the given user). The metabolic signature also varies over time based on metabolic adaptation that occurs, such as computed by the metabolic adaptation calculator 1190.

FIG. 12 depicts a graph 1200 showing an example of calories burned per minute as a function of time over period of time (e.g., a day). Similar data can be provided for extended consecutive periods of time (e.g., multiple days and weeks), such as based on minute to minute energy expenditure data acquired by wearable body sensors. In the example of FIG. 12 the calories burned per minute represent sensor data (e.g., 140) corresponding to minute by minute metabolic rate data acquired by a wearable sensor (e.g., sensor 400 of FIG. 4). The graph includes several different types of activity periods, such as, for example, a sleep (e.g., non-REM sleep, REM sleep) period 1202, active periods (e.g., corresponding to time intervals when cal/min is greater than a high activity threshold, such as about 1.0), recovery periods (following sustained high activity periods) and other sedentary periods (e.g., corresponding to time intervals when cal/min is less than the high activity threshold).

As a further example, FIG. 13 illustrates an example graph 1300 depicting a user's activity over time where activity thresholds are employed to determine different activity intervals, such as can be applied to the type of data demonstrated in FIG. 12. The graph 1300 depicts activity level on the vertical axis versus time on the horizontal axis. As previously described, a body motion sensor or other device (e.g., burn rate monitor) can be employed to provide the data corresponding to the amount of activity on the vertical axis. For example, the activity level can correspond to calories per minute, such as disclosed with respect to FIG. 12. A low activity threshold can be employed to detect intervals that are below a predetermined activity threshold (e.g., no motion detected during sleep, low calorie burned during rest). A high activity threshold can be employed to detect intervals (e.g., activity periods) where higher energy is expended by the user. The activity thresholds can be employed to intervals such as a period of rest or a period of sleep (e.g., below low activity threshold), or during a recovery period after user activity (e.g., between high activity threshold and low activity threshold). The recovery period can be detected after user activity exceeds a predetermined high activity threshold for the user followed by a predetermined low activity threshold for the user, for example. The thresholds can be default values from empirical studies or can be determine for a given user based on data collected for the given user over time, and further can be adapted over time as the given user's health condition changes.

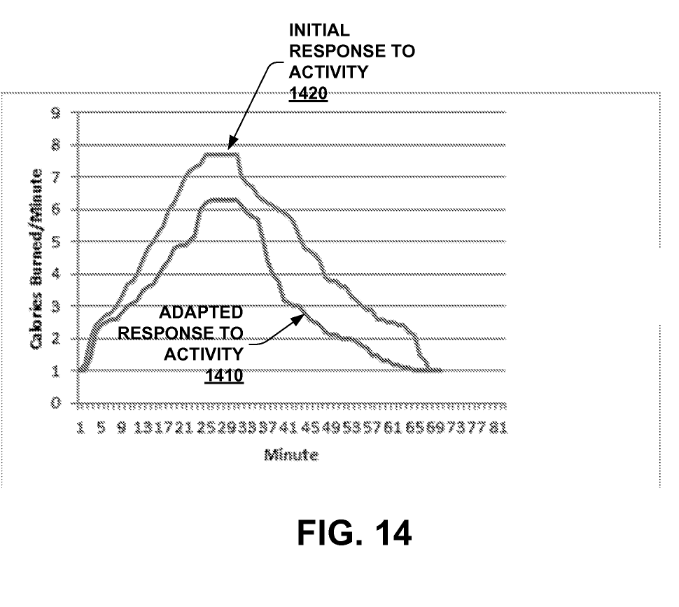

FIG. 14 illustrates an example adaptation that occurs to a given physical activity. In this example, the physical activity is running where basically the same amount of activity is utilized each time a running activity occurs. At 1410, fewer calories are burned per minute during exercise due to adaptation to the exercise compared with the calorie burn rate when the individual began running at a given pace 1420. Recovery (e.g., return to resting metabolism) is also more rapid for the adapted metabolism at 1420. Thus, over time, given the same activity stimulus (e.g., running/exercising approximately the same pace) the body begins to adapt and less calories are burned for the same amount of activity. The systems and methods described herein can detect such adaptation and thus provide recommendations to counter-act the adaptation and thereby improve weight management efficiency.

In view of the foregoing structural and functional description, those skilled in the art will appreciate that portions of the invention may be embodied as a method, data processing system, or computer program product. Accordingly, these portions of the present invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer, or other programmable data processing apparatus (or a combination of devices and circuits) to produce a machine, such that the instructions, which execute via the processor, implement the functions specified in the block or blocks.

These computer-executable instructions may also be stored in non-transitory computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A system, comprising:
a metabolic rate monitor to monitor one or more metabolic determinants to determine a user's metabolic rate;
an interval identifier to detect a plurality of intervals corresponding to a least one type of user activity over a time period, wherein each of the plurality of intervals is employed to record the user's metabolic rate determined by the metabolic rate monitor;
a metabolic adaptation calculator that determines an adaptation of the user's metabolic rate based on analyzing the user's metabolic rate over each of the plurality of intervals; and
a recommendation module to provide an output indicating at least one of the metabolic determinants to adjust in response to the determined adaptation.

2. The system of claim 1, wherein the interval identifier receives sensor data characterizing activity of the user, the interval identifier identifying each interval corresponding to a given type of user activity according to the sensor data relative to at least one predetermined activity threshold.

3. The system of claim 2, wherein the sensor data corresponds to at least one of motions data received from a motion sensor or metabolic rate data from a metabolic rate sensor.

4. The system of claim 2, wherein each identified interval of the given type of user activity includes one of a period of rest, a period of sleep, a high activity period, each interval being identified based on comparing a user activity level, corresponding to the sensor data, relative to at least one threshold.

5. The system of claim 2, wherein each identified interval of the given type of user activity includes a recovery period following user activity, the interval identifier detecting the recovery period as a decreasing activity level between a predetermined high activity threshold for the user and by a predetermined low activity threshold for the user.

6. The system of claim 5, wherein the metabolic adaptation calculator determines the adaptation to the user's metabolic rate by comparing the user's metabolic rate during a plurality of recovery time periods, the interval identifier detecting the plurality of recovery time periods based on comparing metabolic rate data to the predetermined high activity threshold and the predetermined low activity threshold.

7. The system of claim 1, wherein the metabolic adaptation calculator determines the adaptation to the user's metabolic rate by comparing the user's metabolic rate during a plurality of different time periods identified by the interval identifier as corresponding to the same type of user activity.

8. The system of claim 1, wherein a calibration protocol is defined for the user to correlate metabolic changes from periods of sleep with activities that occurred during intervals detected when the user is awake.

9. The system of claim 1, further comprising metabolic determinant data corresponding to the metabolic determinants, wherein the metabolic determinant data include raw sensor data, derived data representing the user's current metabolism or metabolic rate, or user input data input by users.

10. The system of claim 9, wherein the metabolic determinants include medication input, laboratory values input, health conditions input, genetic status inputs, environmental inputs, hunger inputs, thirst inputs, sleep inputs, food intake, food composition, volitional activity, non-volitional activity, or mood inputs.

11. The system of claim 9, wherein the interval identifier identifies each of the plurality of intervals based on at least one of the raw sensor data, the derived data or the user input data, wherein metadata specifying each identified type of activity is linked to time-based metabolic rate data that is stored in memory.

12. The system of claim 1, wherein the output further comprises a graphic display of the real time metabolic rate, broken down into metabolic components.

13. The system of claim 1, further comprising a metabolic signature that determines a relationship between the metabolic determinants and one or more energy expenditure components that contribute to a user's unique energy expenditure over time; and
the metabolic adaptation calculator determining the adaptation to the user's metabolism or metabolic rate based on the energy expenditure components from the metabolic signature.

14. A method, comprising:
monitoring, by a processor, one or more metabolic determinants to determine a user's metabolic rate;
determining, by the processor, a plurality of time intervals of user activity based on comparing an indication of user activity relative to at least one activity threshold;
analyzing, by the processor, the user's metabolic rate during each of the plurality of time intervals; and
determining, by the processor, an adaptation to the user's metabolic rate based on comparing the user's metabolic rate from the plurality of intervals to a predetermined adaptation threshold.

15. The system of claim 14, further comprising:
evaluating motion sensor data representing body movement of the user relative to at least one activity threshold to identify low activity periods when the user activity for a given time interval from the plurality of time intervals is below a low activity threshold.

16. The system of claim 15, wherein the identified low activity periods comprises a period of rest or a period of sleep, or a recovery period following user activity.

17. The system of claim 14, further comprising evaluating motion sensor data representing body movement of the user relative to a high activity threshold for the user and a low activity threshold for the user to identify each recovery period following user activity as corresponding to a decreasing activity level between the high activity threshold and the low activity threshold.

18. A system, comprising:
a metabolic signature that determines a relationship between metabolic determinants, corresponding to metabolic determinant data, and one or more energy expenditure components (EEC) that contribute to a user's unique energy expenditure over time;
a metabolic adaptation calculator that monitors the EEC from the metabolic signature to determine an adaptation to the user's metabolism or metabolic rate; and
a recommendations module that generates recommendations in response to the adaptation determined by the metabolic adaptation calculator.

19. The system of claim 18, wherein the metabolic signature is based on at least one classifier to determine the EEC.

20. The system of claim 19, wherein the metabolic determinants include medication input, medical conditions input, laboratory values input, hunger inputs, thirst inputs, genetic status inputs, environmental influences inputs, sleep inputs, food intake, food composition, volitional activity, non-volitional activity, or mood inputs, and
wherein the EEC includes resting energy expenditure, thermogenic effect of eating, non-activity energy thermogenesis, volitional physical activity thermogenesis and recovery.
